(19) United States
(12) Patent Application Publication
(10) Pub. No.: US 2003/0153811 A1
(43) Pub. Date: Aug. 14, 2003

(54) Fluorescence endoscope with inserted/retracted short-pass filter

(75) Inventor: Andreas Muckner, Berlin (DE)

Correspondence Address: RANKIN, HILL, PORTER & CLARK, LLP, 700 HUNTINGTON BUILDING, 925 EUCLID AVENUE, SUITE 700, CLEVELAND, OH, 44115-1405, US

(73) Assignee: OLYMPUS WINTER & IBE GMBH, Hamburg (DE)

(21) Appl. No.: 10/365,191

(22) Filed: Feb. 11, 2003

(30) Foreign Application Priority Data
Feb. 12, 2002 (DE) 202 02 078.9

(51) Int. Cl. A61B 1/06

(52) U.S. Cl. 600/160; 600/476

(57) ABSTRACT

A fluorescence endoscope to view human organs exhibiting zones marked by fluorescence, the endoscope including a light conduit, a lamp, a short pass filter controlled by a timer so as to be selectively movable into and out of the light path between the lamp and the light conduit, a video camera system viewing the image through a stationary long pass filter, and a video-processing unit connected in series with the video camera system. The video processing unit is controlled by the timer and timed to generate time-separate normal and fluorescence images. The video camera system includes a video camera having a frame frequency that is synchronized with the timer. The video processing unit separately processes and prepares the fluorescence images taken when the short pass filter is inserted into the light beam path and the normal images taken when the short pass filter is retracted out of the light beam path, and displays the fluorescence and normal images in congruent superposition on a display.

The present invention relates to a fluorescence endoscope defined in the preamble of claim 1.

Fluorescence endoscopes are used in Photodynamic Diagnosis (PDD), that is, to view fluorescently marked zones in the human body. Illustratively, such an endoscope allows detecting tumorous regions on the wall of the human bladder, said regions having been enriched beforehand with fluorescent markers. This procedure incurs the drawback, which is comprehensively discussed in the pertinent literature, that on one hand the fluorescent image is very weak while on the other hand a normal image in normal colors is required to allow the physician to associate the fluorescent site with its place in the organ. Known fluorescence endoscopes, which, for example, allow alternating between the fluorescence image and the normal image by means of a foot switch, allow only inadequate correlations.

A fluorescence endoscope according to the preamble of claim 1 of the present invention is known from the German patent document 1,953,114 A1. In this design the video camera system consists of two cameras, one of which is fitted with a long-pass filter and is used to view the fluorescence image while the other, filter-less camera takes the normal image. The timer controlling the short-pass filter does process, along separate paths, the fluorescence image of one camera when the short-pass filter is inserted and the normal image of the other camera when the short-pass filter is retracted and in the procedure it adequately amplifies the fluorescence image. The normal and fluorescence images then may be viewed in juxtaposition on a monitor.

This known design suffers from the drawback of requiring two cameras. Additionally, viewing the juxtaposed images while allowing improved identification of the fluorescent zone in the overall organ also demands some skill because the images must be viewed alternatingly in order to attain correlation.

The objective of the present invention is to create a fluorescence endoscope of the above kind which however offers simplified design and improved identification between fluorescence and normal images.

This problem is solved by the features of claim 1.

The design of the invention comprises a single camera of which the frame frequency is synchronized with the insertion/retraction rate of the short pass filter and with the operation of the video processor. Depending on the inserted/retracted state of the short pass filter, the camera generates fluorescence images or normal images. Because of said synchronization, said images may be recognized as being either ones and be processed separately. Following such separate processing, the images are superposed in congruent manner and then are displayed. The display image so generated therefore shows the suitably amplified fluorescence zone directly on the normal image and as a result the topographical identification of the fluorescence-marked zone in the organ ensues automatically. This design of the fluorescent endoscope is much simplified because requiring only one camera. This camera may be black-and-white or preferably color. The lacking extreme blue portion—which is also lacking in the normal image on account of the stationary long pass filter—does not degrade color perception.

The features of claim 2 are advantageous. These features provide an increase in contrast and/or brightness of the fluorescence image and as a result a clear image of the fluorescing zones is achieved in a normal image, the latter being more intense than the fluorescent image. Such features are offered by claim 3, namely by means of the conventional sampling amplification of the weak fluorescence image relative to the more intense normal image.

The invention is shown in illustrative and schematic manner in the drawing.

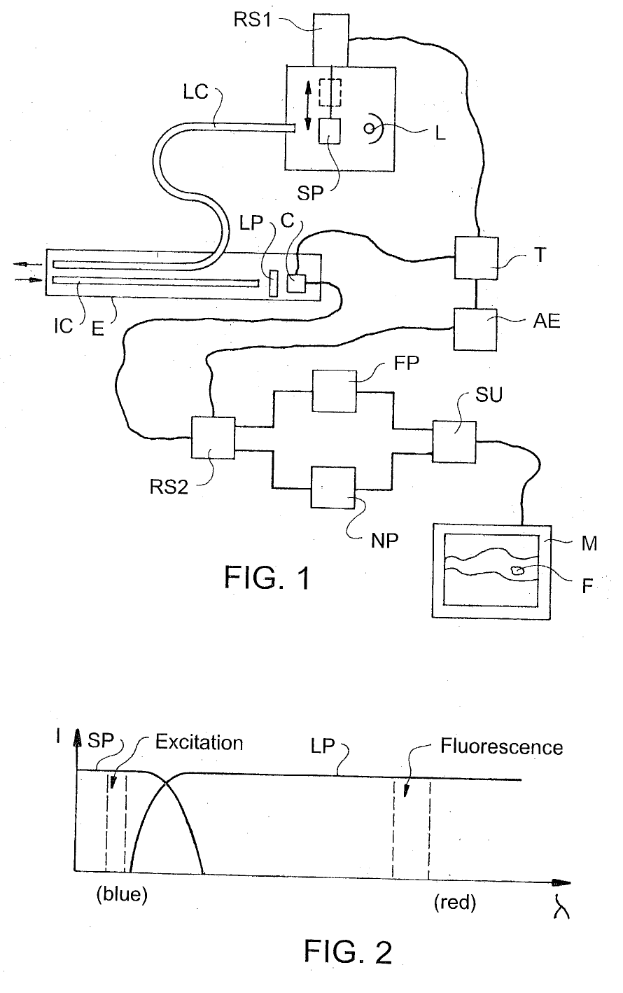

FIG. 1 is a functional block diagram of the fluorescence endoscope of the present invention, and

FIG. 2 is a plot of the transmission curves of the filters used.

FIG. 1 shows an endoscope comprising a housing E receiving an image conduit IC for instance of conventional relay lens design and a light conduit LC at and from which light is received or radiated, resp., in the directions shown by the arrows. The light conduit LC continues outside the endoscope housing E to an illumination means where it is illuminated by a lamp L. A short pass filter SP is mounted between the lamp L and the light conduit LC and can be inserted/retracted by a first reversing switch RS1 in the manner shown into and out of the path of the illumination beam (in the direction of the arrow).

In the shown illustrative embodiment, a camera C is mounted inside the endoscope housing E. This camera for instance is a color CCD chip. A long pass filter LP is mounted between the camera C and the image conduit IC.

FIG. 2 shows the transmission curves of both filters SP and LP which are plotted as the transmission intensity as a function of light wavelength λ. As shown in parentheses, the range of wavelengths goes from blue to red.

As shown in FIG. 2, the stationary long pass filter LP always suppresses very short (blue) wavelengths while transmitting the residual light spectrum of the white light emitted by the lamp L.

When inserted by the first reversing switch RS1, the short pass filter SP only transmits short-wave light while suppressing all longer wavelengths.

When the short pass filter SP is inserted as in FIG. 1 (solid lines), then, as shown in FIG. 2, both filters shall be mutually superposed and as a result light emitted by the lamp L and reflected from the body tissue cannot be seen by the camera C.

If there is a fluorescing site in the viewed tissue zone, for instance a tumor previously marked with fluorescent substances, then the fluorescence will be excited at a short wavelength (blue). A typical range of excitation is denoted by “excitation” in dashed lines in FIG. 2. This fluorescence entails light emission in the red range, namely in the dashed-line “fluorescence” range. The camera is able to detect the fluorescing light and, provided the filters are positioned as in FIG. 2, the image appears before a wholly dark background.

If, as shown in FIG. 1, the short pass filter SP is retracted, then the tissue will be illuminated with white light and the camera C can reproduce the organ through the full range of the long pass filter LP except for the blue range wherein the excitation takes place, that is, with a light discoloration in the extreme blue range.

Accordingly, by inserting and retracting the short pass filter SP, the camera C will alternatingly see fluorescence images (fluorescence against a dark background) and normal images (full light spectrum).

By means of the shown cable, the camera C is synchronized with a timer T which also controls the first reversing switch RS1 to drive the short pass filter SP. In this manner the images from the camera C may be related to the state of the short pass filter SP and hence it is possible to determine whether the camera provides fluorescence images or normal images.

The camera C transmits the image data through the shown cable to a second reversing switch RS2 controlled from the timer T through an adjusting element AE. Accordingly images may be fed by means of the second reversing switch RS2 through the two output cables in such manner that only one of these output cables, namely that connected to a shown fluorescence image processing unit FP, shall be loaded with fluorescence images while that connected to a shown normal image processing unit NP shall be loaded only with normal images.

The fluorescence images processing unit FP is designed to substantially amplify the contrast and/or brightness of the fluorescence image which per se is much weaker than the normal image and to match said fluorescence image's parameters to those of the normal image. The shown output cables of the processing units FP and NP feed a superposition unit SU connected by the shown cable to a monitor M displaying the images. The superposition unit SU superposes in congruent manner the images from FP and NP and makes them available for display on the monitor M. Accordingly said monitor displays a superposition image of normal and fluorescent images. A fluorescent site F on the tubular organ of FIG. 1 may be displayed in a precise correlation.

The fluorescence images processing unit FP in particular may be designed to sum several sampled fluorescence images in order to attain thereby amplification and in particular higher contrast. Moreover noise is also reduced thereby. The fluorescence image so prepared then may be superposed on a normal image in the superposition unit SU. The timing relation between the number of normal and fluorescence images to be processed may be adjusted for instance by means of the adjusting element AE by appropriately driving the first reversing switch RS1 as a function of the timer T.

1. A fluorescence endoscope (E) to view human organs exhibiting zones (F) marked by fluorescence, comprising a light conduit (LC) used for illumination and of which the proximal end is illuminated from a lamp (L) emitting white light passing through a short pass filter (SP) which may be controlled by a timer (T) to be moved into the beam path, further comprising a video camera system (C) viewing the image through a stationary long pass filter (LP), further a video-processing unit (RS2, FP, NP, SU) connected in series with the video camera system and controlled by the timer (T) and timed to generate time-separate normal and fluorescence images,
characterized in that
the video camera system comprises a video camera (C), the frame frequency of which is synchronized with the timer, where the video processing unit (RS2, FP, NP, SU) separately processes and prepares the fluorescence images taken when the short pass filter (SP) is inserted and the normal images taken when the short pass filter is retracted and then displays said fluorescence and normal images in congruent superposition on a display (M).

2. Fluorescence endoscope as claimed in claim 1, characterized in that the video processing unit (FP) amplifies the contrast and/or brightness of the fluorescence images.

3. Fluorescence endoscope as claimed in claim 3, characterized in that the video viewing unit (VP) each time buffers and superposes several fluorescence images and in that the amplified fluorescence image so prepared is superposed on a normal image.
